(19) United States
(12) Patent Application Publication
(10) Pub. No.: US 2024/0343698 A1
(43) Pub. Date: Oct. 17, 2024

(54) Bioderived monomers for the generation of polybenzoxazine thermosets and thermoplastics

(71) Applicant: Alliance for Sustainable Energy, LLC, Golden, CO (US)

(72) Inventors: Caroline Bradshaw HOYT-ANDERSON, Jacksonville, FL (US); Gregg Tyler BECKHAM, Golden, CO (US); Nicholas A. RORRER, Golden, CO (US)

(21) Appl. No.: 18/623,312

(22) Filed: Apr. 1, 2024

Related U.S. Application Data
(60) Provisional application No. 63/493,364, filed on Mar. 31, 2023.

(51) Int. Cl. C07D 265/12; C08G 73/02

(52) U.S. Cl. CPC C07D 265/12; C08G 73/0233

(57) ABSTRACT

Described herein is the bio-derivation of the functional replacement from biological conversion which addresses the need to replace petroleum-derived materials from caustic processes. Current technologies do not address the end-of-life for plastics, and the incorporation of reversible chemistry for high performance materials would enable a circular material life cycle. The diamine in the polybenzoxazine utilizes a bioderived material with an additional functional handle to polymerize linearly and crosslink for thermal and mechanical properties comparable to petroleum derived materials.

### CROSS-REFERENCE TO RELATED APPLICATIONS

This application claims priority from U.S. Provisional Patent Application No. 63/493,364 filed on Mar. 31, 2023, the contents of which are incorporated herein by reference in their entirety.

### CONTRACTUAL ORIGIN

This invention was made with government support under Contract No. DE-AC36-08GO28308 awarded by the Department of Energy. The government has certain rights in the invention.

### BACKGROUND

Polynaphthoxazines and polybenzoxazines have emerged as an important class of thermomechanically robust thermosets to displace phenolic resins because of the molecular tunability paired with near-zero shrinkage and the absence of volatile product formation during curing; however, they still require higher cure temperatures and long cure times. Thus, reducing the cure temperatures and times for these thermosets is desirable as it may expand their applications and reduce their manufacturing intensity.

### SUMMARY

Described herein is the bio-derivation of the functional replacement from biological conversion which addresses the need to replace petroleum-derived materials from caustic processes. Current technologies do not address the end-of-life for plastics, and the incorporation of reversible chemistry for high performance materials would enable a circular material life cycle. The diamine in the polybenzoxazine utilizes a bioderived material with an additional functional handle to polymerize linearly and crosslink for thermal and mechanical properties comparable to petroleum derived materials.

The present application provides biological conversion of sugars and lignin into biobased monomers that are incorporated into both thermosets with triazine networks consisting of polybenzoxazines and as thermoplastic additives into various polymers for additional thermal and mechanical property improvements. For example, the incorporation of 4-aminophenyl ethyl-R monomers as the amine component of benzoxazine synthesis into these polymers provides a functional handle in the benzoxazine for further modifications. The use of the 4-aminophenyl ethylamine (4-APEAm) provides a point of reversibility in the triazine network. In an alternative use for 4-APEAm, the amine can be used to incorporate the benzoxazine monomer into various resins to improve thermal and mechanical properties. The use of lignin derived monomers is an alternative phenolic unit to build a benzoxazine from with thermal and mechanical property improvements not directly reliant on petroleum products.

In an aspect, provided is a method comprising: a) providing a bio-derived aromatic amine; b) reacting the bio-derived aromatic amine with a phenol to generate a naphthoxazine. The method may include polymerizing the naphthoxazine to form a polybenzoxazine.

The polymerization step may be performed via known chemical methods, including cationic ring opening polymerization or amine protection/deprotection where the aromatic amine or naphthoxazine contains a nitrogen protecting group.

Importantly, the step of reacting, polymerization or crosslinking may be performed at lower temperatures that similar petroleum derived polymers. The step of reacting may occur at a temperature less than or equal to 250° C., 200° C., 175° C., 150° C., or optionally, 125° C. Additionally, the reaction temperature for the polymerization or crosslinking reaction may occur at less than or equal to 300° C., 250° C., 220° C. or optionally, 200° C. The step of polymerization may be performed in the presence of formaldehyde.

The phenol may be a bio-based phenol, for example, naphthol, resveratrol, resorcinol, cardanol, guaiacol, eugenol, thymol, vanillin, sesamol, terpenediphenol, magnolol, daidzein, naringenin, urushiol, functionalized coumarin, naringenin, apigenin or a combination thereof.

The aromatic amine may be generated microbially from sugar or derived from biomass, such as lignin. The aromatic amine may be described by the formula:

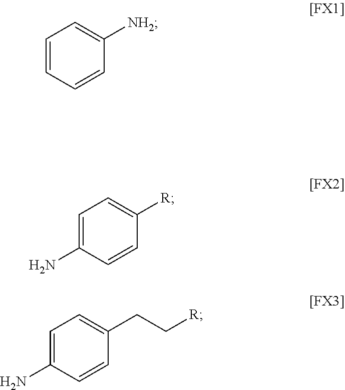

or a combination thereof; wherein R is a nitrogen protecting group comprising an alcohol, a carboxylic acid, BOC (tert-butyloxycarbonyl), a phthalic anhydride, a tehtrachlorophthalic anhydride, or a combination thereof.

### BRIEF DESCRIPTION OF DRAWINGS

Some embodiments are illustrated in referenced figures of the drawings. It is intended that the embodiments and figures disclosed herein are to be considered illustrative rather than limiting.

FIG. 1 provides an example scheme for reacting naphthoxazine to yield crosslinked polynaphthoxazine.

FIG. 2 provides bio-based phenolic monomers and amine monomers previously described in aromatic oxazine synthesis, where the box (lower left) illustrate the monomers described herein.

FIG. 3 illustrates the synthesis of partially bio-based naphthoxazine with a bio-based amine and the resulting polynaphthoxazine.

FIG. 4 provides an example scheme for generating napthoxazine.

FIG. 5 illustrates the HSQC spectrum of molecule 2a, as described in FIG. 4.

FIG. 6A provides the ¹H NMR spectrum of molecule 2a, as described in FIG. 4, in DMSO-d₆.

FIG. 6B provides the ¹³C NMR spectrum of molecule 2a, as described in FIG. 4, in DMSO-d₆ at 25° C.

FIG. 7 provides a DSC thermogram of 2b compared to 2c and 2a (as shown in FIG. 4) at a heating rate of 5° C./min.

FIG. 8 shows DSC thermograms of 2a (as shown in FIG. 4) at heating rates of 2, 5, 10, 15, 20° C./min.

FIG. 9 provides a plot of the Kissinger method for calculating activation energy of polymerization.

FIG. 10 provides a plot of the Ozawa method for calculating activation energy of polymerization.

FIG. 11 shows FT-IR spectra after heating 2a for 30 minutes at 30, 50, 80, 110 and 140° C.

FIG. 12 illustrates the conversion of oxazine ring-opening at 130° C. as a function of time.

FIG. 13 provides DSC thermograms of 2a pretreated to 30, 60 and 100° C. for 1 hr with a heating ramp of 5° C./min.

FIG. 14 provides thermograms of molecule 2a added to 2b at concentrations of 5, 10 and 25 mol % and heated at 5° C./min.

FIG. 15 provides an example scheme for the amine protection/deprotection polymerization reaction of aromatic amines to form polynaphthoxazine.

FIG. 16 provides an example scheme for the amine protection/deprotection polymerization reaction of aromatic amines to form polynaphthoxazine.

FIG. 17 provides the effect of curing temperature on heat input required where negative heat flows do not require energy via modelling of polynaphthoxazine production.

FIG. 18 shows total energy input required for the cure as a function of cure temperature via modelling of polynaphthoxazine production.

### DETAILED DESCRIPTION

The embodiments described herein should not necessarily be construed as limited to addressing any of the particular problems or deficiencies discussed herein. References in the specification to “one embodiment”, “an embodiment”, “an example embodiment”, “some embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein the term “substantially” is used to indicate that exact values are not necessarily attainable. By way of example, one of ordinary skill in the art will understand that in some chemical reactions 100% conversion of a reactant is possible, yet unlikely. Most of a reactant may be converted to a product and conversion of the reactant may asymptotically approach 100% conversion. So, although from a practical perspective 100% of the reactant is converted, from a technical perspective, a small and sometimes difficult to define amount remains. For this example of a chemical reactant, that amount may be relatively easily defined by the detection limits of the instrument used to test for it. However, in many cases, this amount may not be easily defined, hence the use of the term “substantially”. In some embodiments of the present invention, the term “substantially” is defined as approaching a specific numeric value or target to within 20%, 15%, 10%, 5%, or within 1% of the value or target. In further embodiments of the present invention, the term “substantially” is defined as approaching a specific numeric value or target to within 1%, 0.9%, 0.8%, 0.7%, 0.6%, 0.5%, 0.4%, 0.3%, 0.2%, or 0.1% of the value or target.

As used herein, the term “about” is used to indicate that exact values are not necessarily attainable. Therefore, the term “about” is used to indicate this uncertainty limit. In some embodiments of the present invention, the term “about” is used to indicate an uncertainty limit of less than or equal to ±20%, ±15%, ±10%, ±5%, or ±1% of a specific numeric value or target. In some embodiments of the present invention, the term “about” is used to indicate an uncertainty limit of less than or equal to ±1%, ±0.9%, ±0.8%, ±0.7%, ±0.6%, ±0.5%, ±0.4%, ±0.3%, ±0.2%, or ±0.1% of a specific numeric value or target.

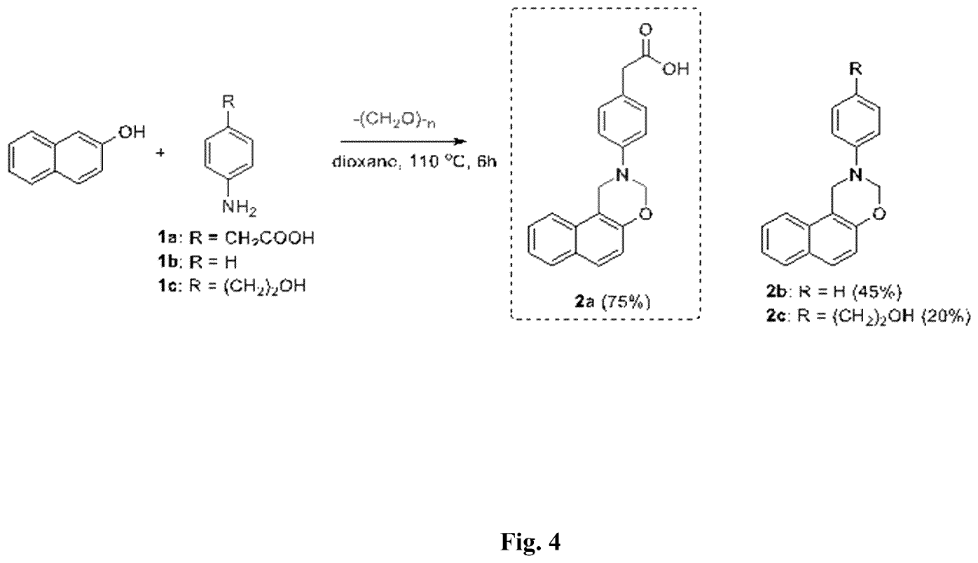

As used herein, “2a” and “molecule 2a” refers to 2-(4-(1H-naphtho[1,2-e][1,3]oxazin-2(3H)-yl)phenyl) acetic acid and illustrated in FIG. 4.

The provided discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. Thus, the following claims are hereby incorporated into this Detailed Description, with each claim standing on its own as a separate aspect, embodiment, or configuration.

### Example 1—Performance-Advantaged Naphthoxazine Synthesis and Effects on Ring-Opening Polymerization Through a Bio-Based Bifunctional Aromatic Amines

Described herein is the synthesis of a naphthoxazine utilizing a bio-derivable amine containing a catalytic carboxylic acid to promote acid-catalyzed ring opening polymerization. The resultant naphthoxazinc reduces the polymerization temperature from 249° C. to 142° C. while still possessing stability at elevated temperatures for extended period of time. Furthermore, the bio-derivable naphthoxazine can be used as an additive (5 mol %) with both a control naphthoxazine, as well as a bisphenol-A benzoxazine, to reduce the cure temperature by of 40 and 50° C. respectively. The reduction in energetic costs associated with this bio-derivable additive have been highlighted through process analysis which demonstrate a reduction in processing energy of 24%.

### Introduction

Polybenzoxazines and polynaphthoxazines are alternative materials to phenolic resins that are composed of a phenol, primary amine, and formaldehyde, which undergo a Mannich reaction to form the benzoxazine ring. Typically, benzoxazine-containing materials exhibit high thermal resistance and low flammability, near-zero shrinkage during curing, low dielectric constant, and molecular flexibility and tunability ideal for a variety of applications. These desirable material features have led to electronic and aerospace industry interest. However, the superior thermomechanical properties that polybenzoxazines exhibit typically require elevated ring-opening polymerization temperatures of the benzoxazine monomers, in turn leading to energy-intensive curing conditions. Indeed, many polybenzoxazines are produced through thermally-activated cationic ring-opening polymerization (ROP), which occurs over a temperature range of 200-280° C. The temperatures required to polymerize can overlap with degradation temperatures of the materials and contributes to a higher energy consumption for synthesis of these materials.

One approach to decreasing the cure temperatures has been by investigating the molecular flexibility of benzoxazine structures through both structural and functional placement of catalytic moieties. Previous literature has identified electron-withdrawing and electron-donating substitutions to lower polymerization temperatures para- to the amine of the benzoxazine ring. Functionalizing the primary amine has been demonstrated through pendent functionality including acetyl- and allylamines, alkylamines, fused rings, cyanate esters, and aniline as the classic aromatic amine. Both alcohols and carboxylic acids demonstrated the lowering of polymerization temperatures by as much as 100° C., due to the acidic proton, which inevitably has been highlighted in a few earlier examples with initial benzoxazines to lower the temperature.

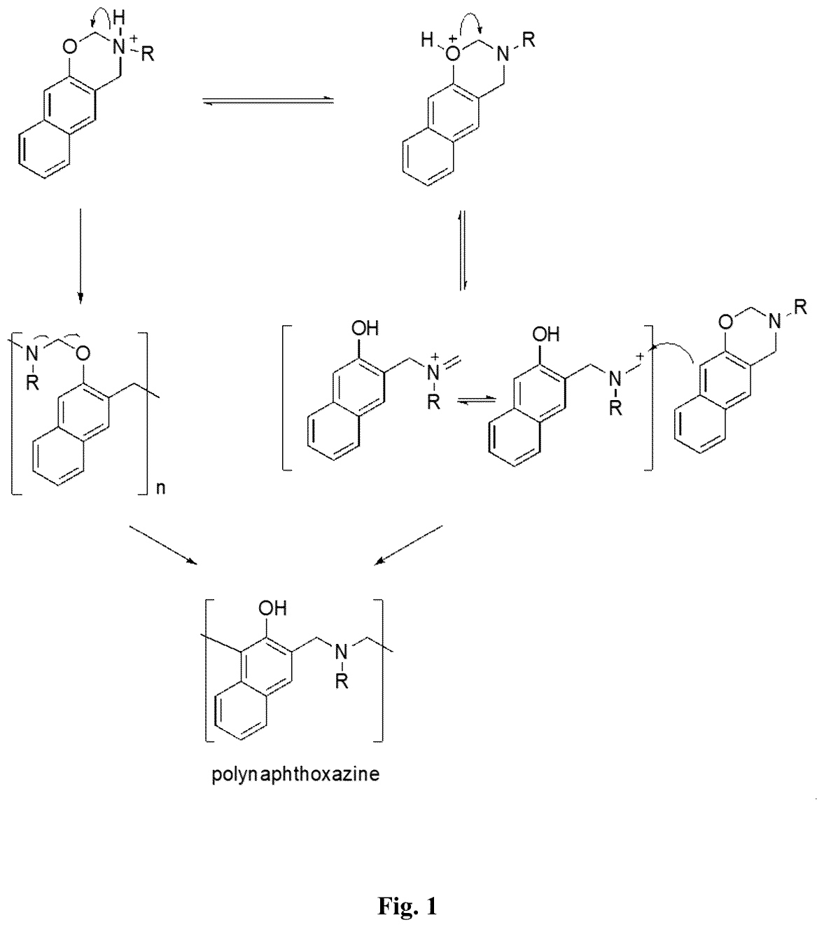

As shown in FIG. 1, the polymerization of benzoxazine monomers has been demonstrated to proceed via a thermally induced or an “initiated” acid-catalyzed cationic ring-opening process. Stabilization of the cationic species can occur on both the nitrogen and oxygen, with a rearrangement of the aryl ether to a phenolic structure or formation of the iminium species which tautomerizes and undergoes electrophilic aromatic substitution. Due to the elevated temperatures necessary for the thermally induced cationic initiation, there is a desire to develop catalysts or catalytic benzoxazine motifs with reduced curing temperatures, which would reduce energy consumption and expand the application of these materials. Various external catalysts have reduced the curing temperature, such Lewis acid catalysts with the addition of solvent, down to 120-160° C. Fourth period-acetylacetonato transition metal complexes exhibited onset ROP temperatures of 120° C.; however, the highly active nature of these catalysts initiated ROP during storage and lead to increased viscosity of formulations. Thus, improving the stability of the benzoxazine solutions has been developed through latent catalytic moieties. Several prominent examples from Ishida and Froimowicz have incorporated latent-catalytic functionality into the structures through hydrogen-bonding motifs in an asymmetric naphthoxazine and more recently using bio-sourced naringenin as the phenolic component.

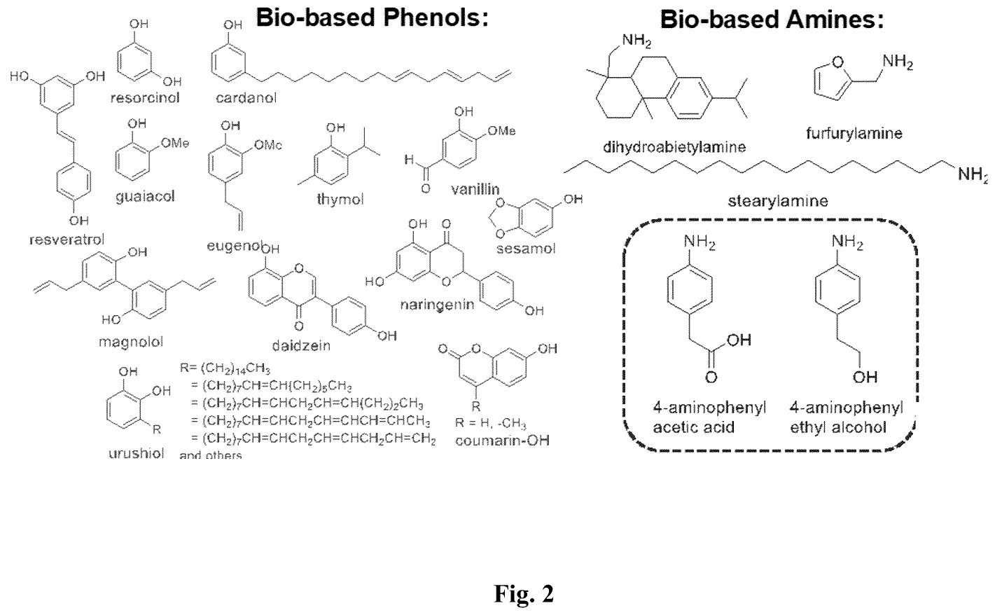

Recent efforts to employ alternative biomass-based raw materials to replace petroleum monomers in polybenzoxazine syntheses have been successful for the phenolic monomer, but limited examples for the amine-containing monomer. The relative case to access bio-based compounds with a phenolic —OH functionality, shown in FIG. 2, has drawn significant interest for benzoxazine synthesis over the past decade with examples such as terpenediphenol, sesamol, cardanol, resorcinol, guaiacol, eugenol, resveratrol, magnolol, thymol, vanillin, urushiol, functionalized coumarin, daidzein, naringenin, and apigenin. To date, there are fewer examples of bio-based amine monomers, such as dehydroabietylamine, furfurylamine and stearylamine. Thus, there is an opportunity to continue increasing the bio-based content in these materials. Recently, aromatic amine compounds, 2-(4-aminophenyl) acetic acid (APAA) and 2-(4-aminophenyl) ethanol were produced microbially from sugars and due to both structure and functionality, presented as valuable naphthoxazine components possessing catalytic functionality to be exploited.

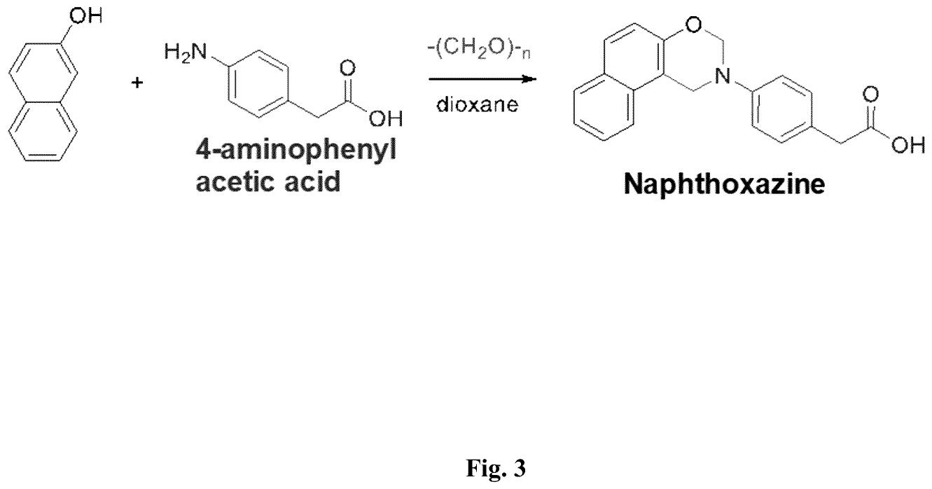

Described herein and as shown in FIG. 3, is synthesis and evaluation a series of catalytic naphthoxazines with dual functionality from bio-based aromatic amines, and study of the monomer structures by ¹H NMR spectroscopy, ¹³C NMR spectroscopy, differential scanning calorimetry (DSC), and Fourier transform infrared (FT-IR) spectroscopy. The resulting polynaphthoxazines were analyzed by thermogravimetric analysis (TGA) as well as FT-IR spectroscopy. The naphthoxazines were analyzed as both a pure material and as an additive with a control naphthoxazine and a bisphenol-A based benzoxazine (BPA-a), which exhibited lower polymerization temperatures at low mole percentages. Furthermore, the reduction in cure temperature was modeled using conversion kinetics to fundamentally quantify the impact of improved processability which showed a 29% reduction in the energy input for curing. Additionally, we modeled the production of the bio-based amine in the naphthoxazine system which highlights the economic and sustainability aspects of production at scale.

### Results

Naphthoxazine Monomer Synthesis. The synthesis for the naphthoxazines shown in FIG. 4 was adapted to accommodate the solubilization of the bio-based amines, both 2-(4-aminophenyl) acetic acid (1a) and 2-(4-aminophenyl) ethanol (1c). Generally, naphthols are more reactive due to the electron-rich aromatic rings and have demonstrated robust thermal behavior with polyfunctional naphthoxazines. It has been shown that with naphthol as the phenolic component and a fused ring system improved the thermal properties in the monofunctional system, thus naphthol was chosen to synthesize the monofunctional naphthoxazine with the bio-based aromatic amines to evaluate the catalytic functionality. The synthesis was carried out in a one-pot Mannich reaction, with resulting reactions cooled to room temperature and 2a and 2c precipitated from solution and recrystallized to yield yellow powders of 75% and 20%, while 2b was further purified through column chromatography. 2b after purification was a dark orange, viscous oil at 45% yield.

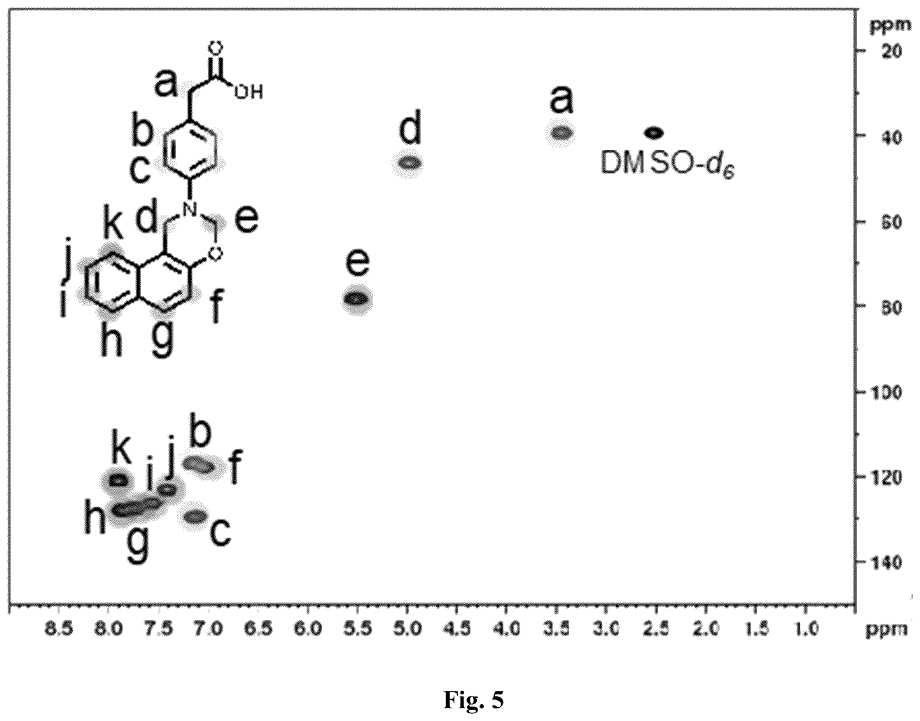

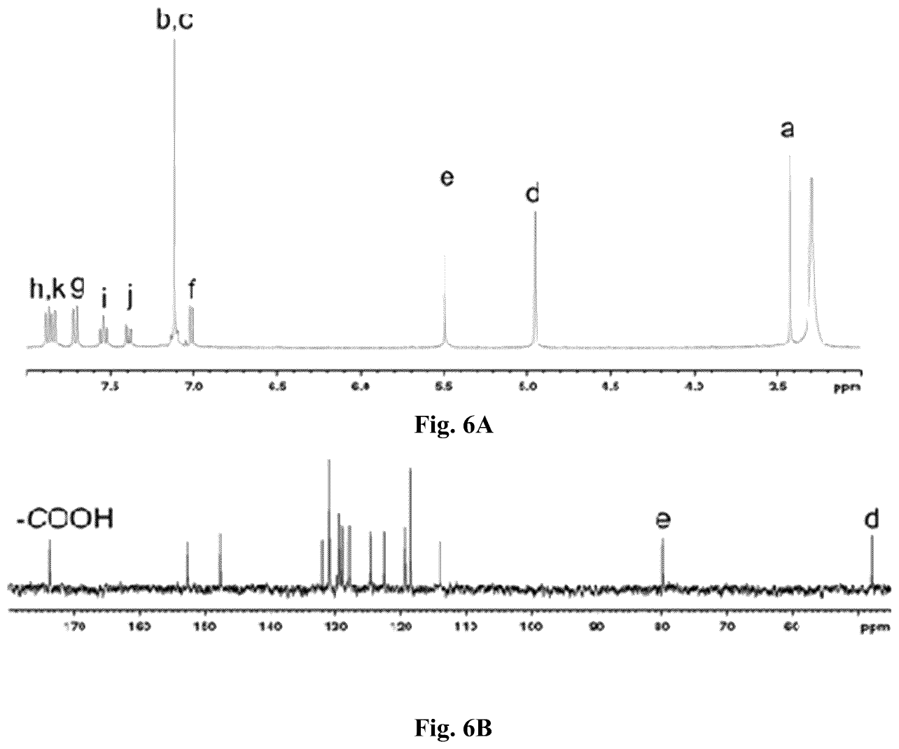

FIGS. 5-6 show the structural characterization for the three naphthoxazines using ¹H, ¹³C, and HSQC spectroscopy to verify pure product was formed with no remaining isomers. In FIGS. 5-6 the identification of clear singlets from the Ar—CH₂—N at 4.96 ppm and the N—CH₂—O at 5.50 ppm are indicative peaks from the formation of a benzoxazine ring. Further identifying the benzoxazine formation can be seen in the aromatic splitting pattern below in FIG. 6. If the formation of the benzoxazine ring were to occur in the f- and g-labelled positions of the naphthol ring, there would be two singlets in the aromatic region corresponding to the neighboring quaternary carbons on either side of the aromatic protons. However, in this case, we observe two doublets at 7.02 ppm (f) and 7.71 ppm (g) with corresponding ¹³C signals at 119.3 ppm and 128.1 ppm. HSQC spectra of both 2c and 2d display signature benzoxazine peaks at similar shifts as well as further benzoxazine ring vibrations observed in the FT-IR spectra.

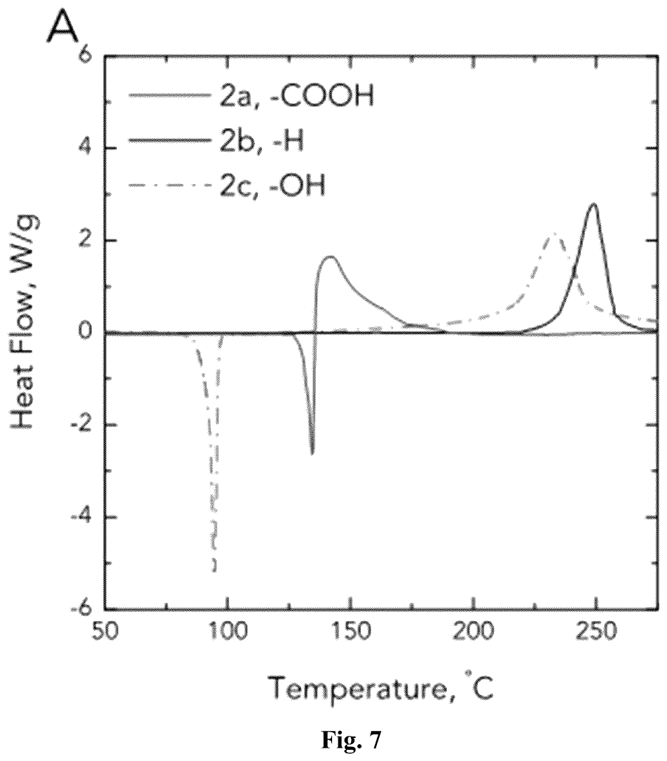

Polymerization of Naphthoxazines via Thermal Methods. The curing behavior of the three naphthoxazines was investigated by DSC, as shown in FIG. 7. The exotherms of 2a, 2b, and 2c represent the maximum polymerization temperature (Tₚ) of each naphthoxazine. The control 2b displays an exotherm around 249° C. which is a typical curing temperature for this benzoxazine structure. The addition of catalytic moieties covalently incorporated into the bio-based aromatic amine was explored by the thermal characterization of both a naphthoxazine with a primary alcohol and carboxylic acid. 2c is a solid pale-yellow powder with a melting point demonstrated by the sharp endothermic peak at 95° C. and the ring-opening of the benzoxazine at 234° C. 2a is also a light-yellow solid which displays a sharp melting endotherm at 134° C. but is quickly polymerized with an exotherm peak at 142° C. Though 2c does display a slight decrease in the polymerization exotherm temperature, the carboxylic acid moiety demonstrates a larger reduction in the ring-opening polymerization temperature of the naphthoxazine.

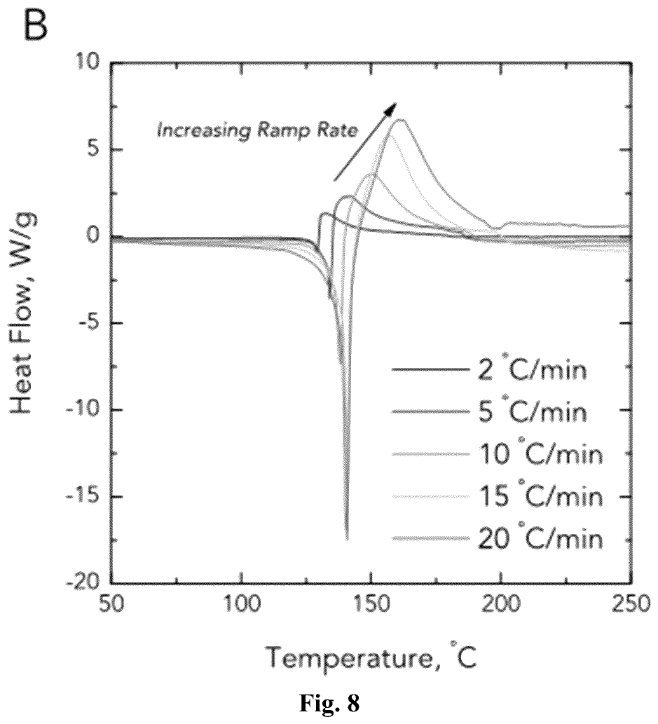

The polymerization kinetics were studied through nonisothermal DSC heating rate of 2, 5, 10, 15, and 20° C./min heating ramps as shown in FIG. 8. The polymerization temperature exotherm peak, Tₚ, increases with increasing heat rates as to be expected. The activation energy of the polymerization was calculated via the Kissinger and Ozawa methods. The Kissinger method is based on Equation 1:

$$ln ( \frac{β}{{T}_{p}^{2}} ) = ln \frac{AR}{{E}_{a}} - \frac{{E}_{a}}{{RT}_{p}}$$

where β is the heating ramp, Tₚ is the polymerization temperature designated from the peak of the polymerization exotherm, A is the frequency factor, R is the gas constant and Ea is the activation energy. The Ozawa method activation energy is calculated by Equation 2:

$$ln ( β ) = C - 1.052 \frac{{E}_{a}}{{RT}_{p}}$$

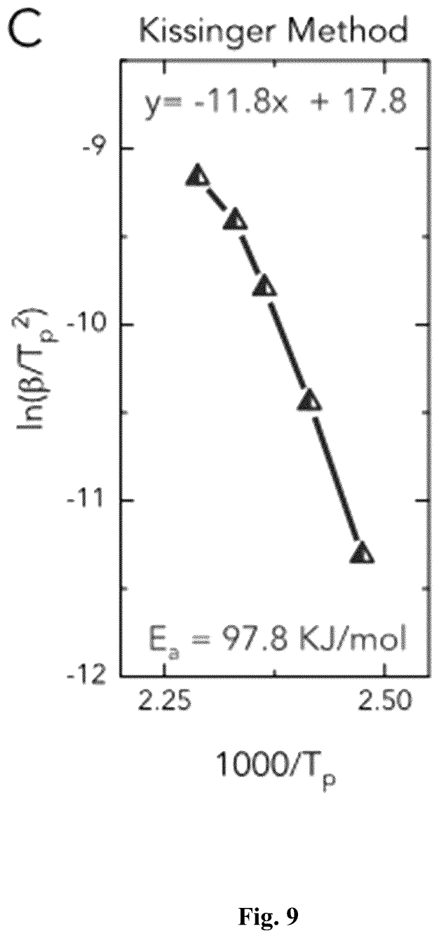

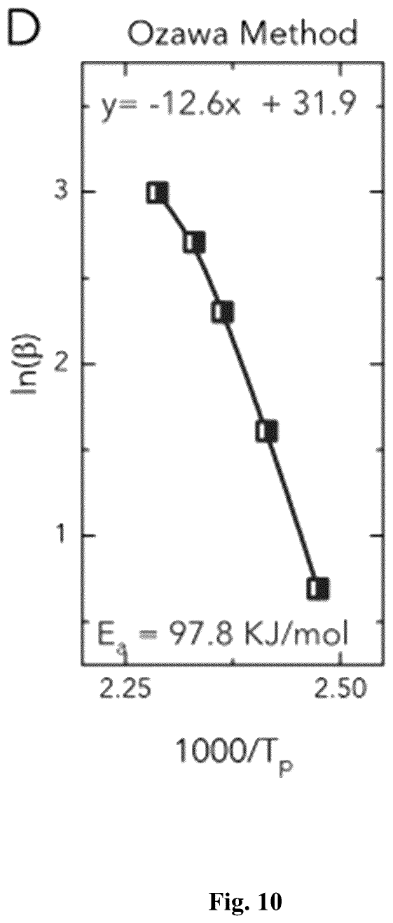

with C being a constant. By plotting the data obtained with ln(β/Tₚ²) or ln(β) on the y-axis as a function of 1/Tₚ, the activation energy can be calculated from the slope of the linear plotted data as shown in FIGS. 9-10. According to the data collected by both methods, the activation energy for polymerization of 2a is 97.8 and 99.5 kJ/mol. The linear nature of both ln(β) and ln(β/Tₚ²) as a function of 1/Tₚ is indicative of a single polymerization mechanism which follows previously proposed acid-catalyzed ring opening polymerizations.

The cure temperature for 2a resides between two recent examples of catalytic benzoxazines with latent catalytic functionality built into the system. An asymmetric naphthoxazine synthesized with a hydrogen-bonding motif exhibited activation energies calculated through these two methods of 107.2 and 109.2 kJ/mol while a naringenin-based benzoxazine produced similar DSC thermogram characteristics to the naphthoxazine produced here and activation energies slightly lower of 93.1 and 94.8 kJ/mol. The estimated activation energy of the monofunctional naphthoxazine 2a is comparative to a monofunctional benzoxazine with a reported Ea by the Ozawa method of 96 kJ/mol and much lower than a classic benzoxazine at 116 kJ/mol. The Ea of 2a is well below that of other benzoxazines with amide and imide functionality of 167 and 247 kJ/mol. This result indicates that 2a is readily activated to polymerize. The advantage of incorporating carboxylic acid functionality covalently to the structure and the stability of the bulk material will be discussed further herein.

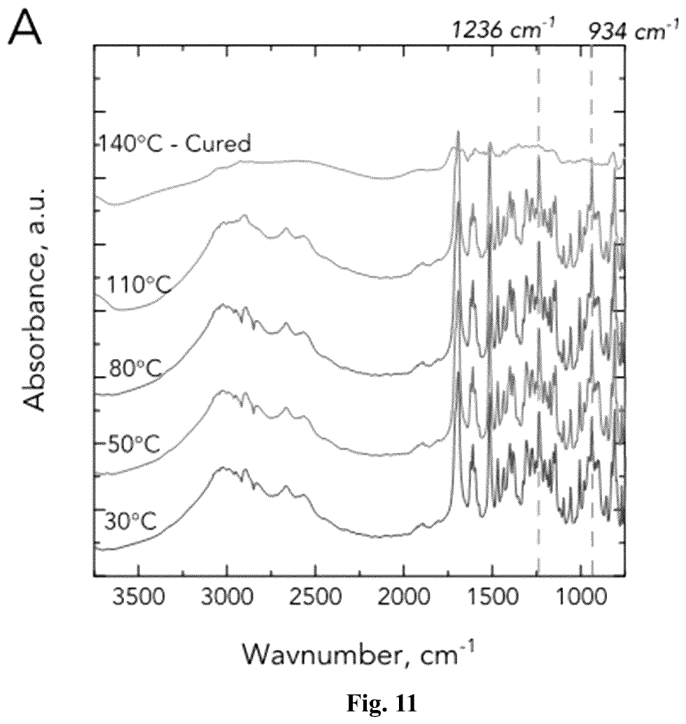

Further investigation into the polymerization behavior of 2a was investigated through FTIR spectroscopy as shown in FIG. 11. To carry out this analysis, the naphthoxazine monomer was heated to each temperature individually for 30 minutes and analyzed by FTIR. The reduction in absorbance of both the 934 and 1236 cm⁻¹ stretches associated with the oxazine ring are indicative of the ring opening of the monomer above 110° C. The oxazine ring opening occurs rapidly once the monomer is melted as expected, while prior to melting the oxazine stretches remain largely unchanged. At temperatures past the melting point of 134° C., the C═O stretch at approximately 1693 cm⁻¹ significantly decreased, suggesting the final polymer is decarboxylated and the reactivity of the naphthoxazine as an additive is rapid once the monomer is melted. The overlay of the DSC exotherm with the TGA analysis demonstrates that the ring opening temperature is roughly 35° C. lower than the T_{d5%} at 167° C., leading to the polymerization of the material prior to degradation with initial mass loss likely due to decarboxylation. When the material is polymerized and the polynaphthoxazine is analyzed by TGA, the T_{d5%} is increased to 194° C.

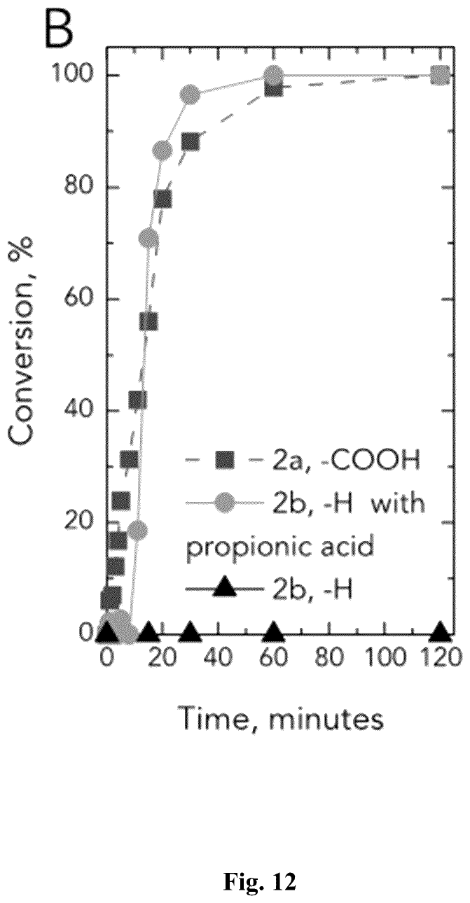

FIG. 12 demonstrates the monomer conversion of the benzoxazine ring opening with insight into the mechanistic behavior of the carboxylic acid at 130° C. The cationic polymerization for benzoxazines has been studied both with thermal activation as well as with initiators such as an acidic proton. The covalently bound acid in 2a was compared to the externally added acidic proton (propionic acid) and monitored for ring-opening kinetics. The polymerization kinetics of 2a compared with 2b and propionic acid reach similar overall conversions of the ring-opened oxazine with an induction period of approximately 10 minutes. The lack of miscibility between 2b and propionic acid could explain the induction period, as this has been observed in previous benzoxazine studies and is not unexpected with the hydrophobic, nonpolar 2b compared to an aqueous acid. In terms of application, the addition of a volatile small molecule acid negates the benefits of no by-product production in the curing of these materials, and supports the inclusion of a catalytic species within the monomer structure with no additional formulation necessary.

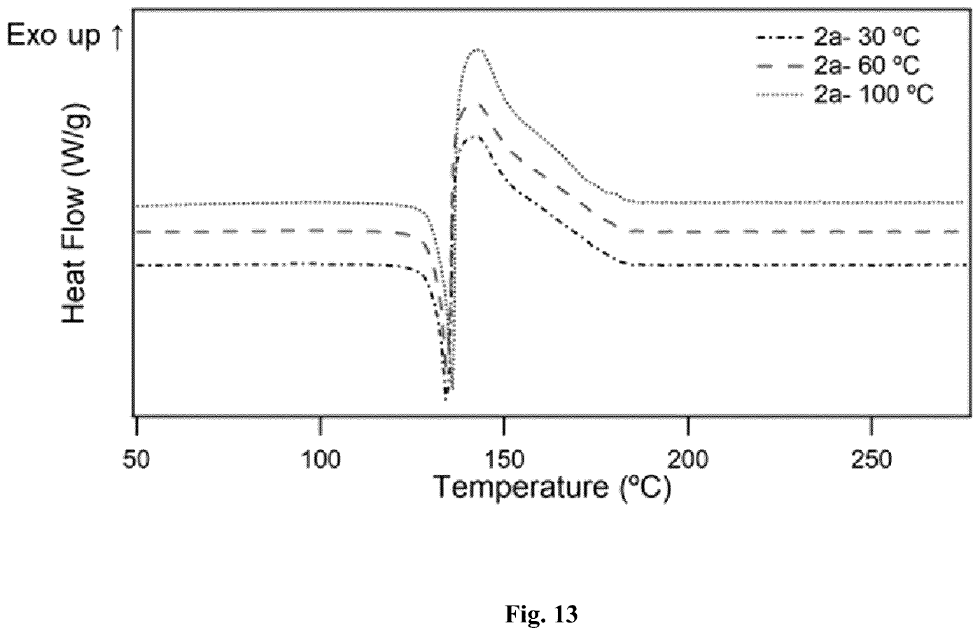

The stability and shelf life of the material was demonstrated in FIG. 13, where over the course of 9 months, and exposure to elevated temperatures the material remained unchanged supporting the stability and short curing times necessary for a polynaphthoxazine material. The stability of the built-in catalytic moiety of 2a was evaluated by analyzing both structural and thermal behavior at various temperatures through heating the compound to various temperatures under the polymerization temperature and examining the polymerization behavior by DSC as well as structural changes through ¹H NMR. The DSC thermograms demonstrate similar polymerization behavior, which indicates the catalytic moiety is stable on these molecules at elevated temperatures. Further, the ¹H NMR of the pretreated 2a samples exhibits no structural changes associated with ring opening polymerization or structural degradation. The lack of structural changes under heated conditions indicates the shelf stability of this naphthoxazine will not be prone to degradation overtime with an acidic moiety present on the structure.

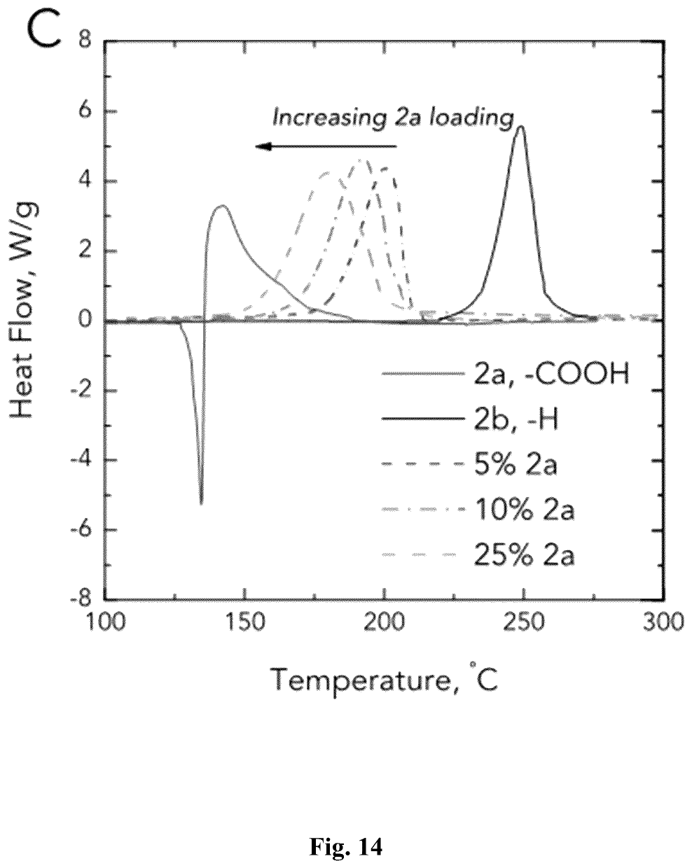

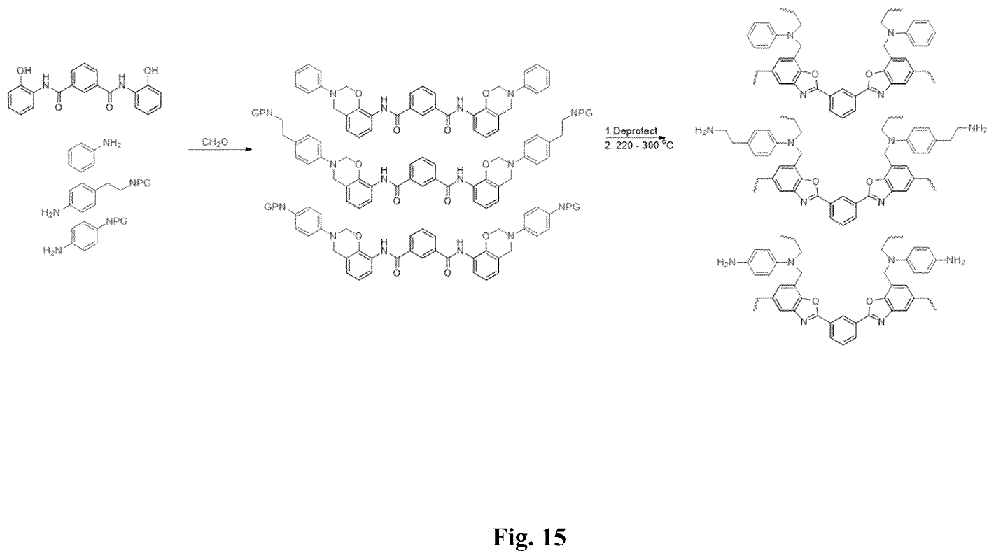

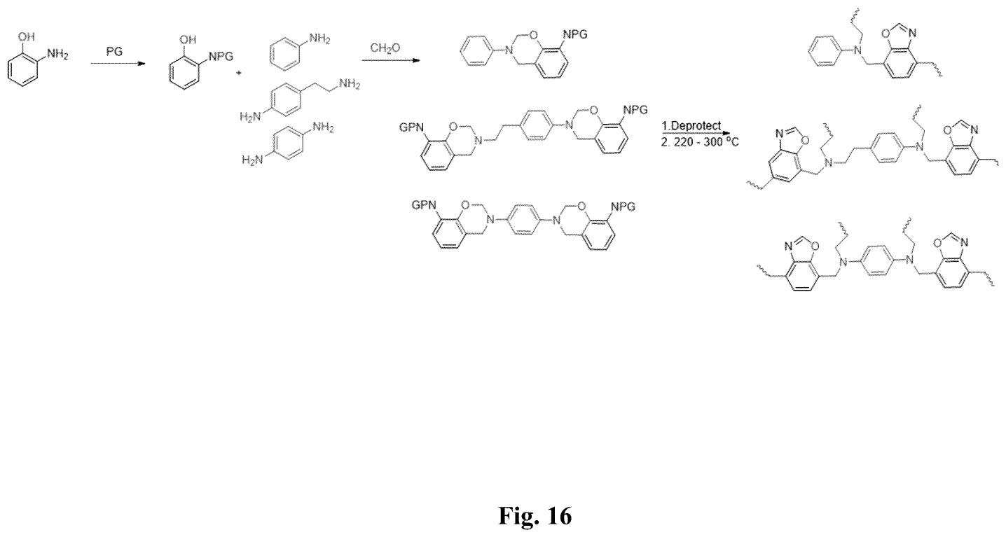

Next, we explored the decreasing of polymerization temperatures through using 2a as an additive with the control 2b shown in FIG. 14. The addition of 5, 10, and 25 mol % 2a was added to 2b and the thermograms display a clear decrease in copolymerization temperatures with increasing 2a mol %. The addition of only 5 mol % 2a, as shown by the green curve, displayed a decrease in copolymerization temperature of roughly 50° C., Tₚ=201° C. from 249° C. Further depression of the copolymerization temperatures was observed at 10 mol % 2a Tₚ=192° C. and 25 mol % 2a Tₚ=181° C., though the effect of added carboxylic acid is less dramatic than the initial addition of catalytic 2a. To be expected, the copolymerization of the 2b with molar additional of 2a displays a thermogram characteristic of 2b rather than displaying a melt temperature of any sort associated with the 2a. Further, 2a was used as an initiator with a bisphenol-A based benzoxazine at 5 mol % and the polymerization temperature was reduced from 245° C. to 206° C. demonstrating the catalytic efficacy of 2a in low concentrations without compromising the thermal properties of the overall polybenzoxazine.

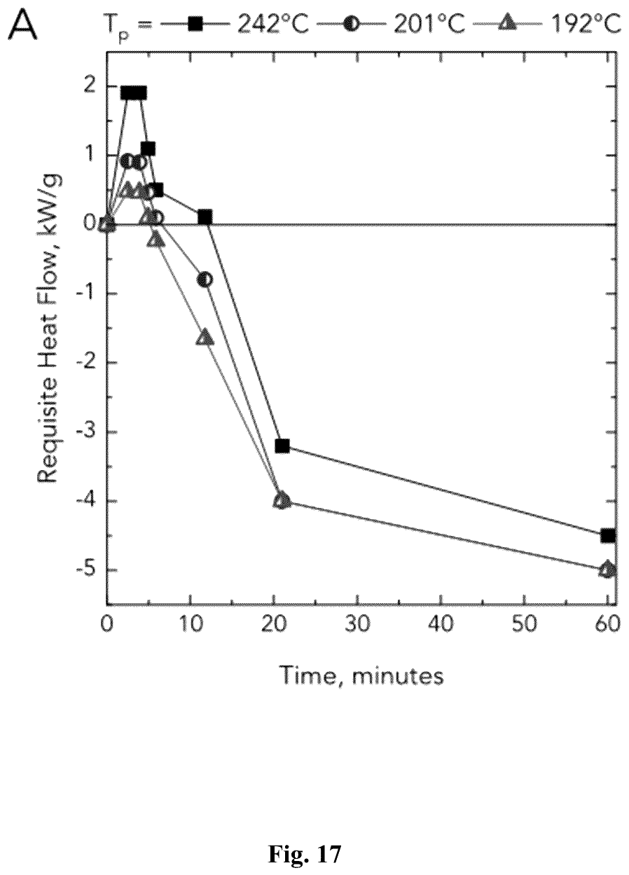

Finally, aligned with the experimental findings which demonstrated that the use of the APAA-naphthoxazine could lower the cure temperature when used as an additive, we investigated the impact of reducing polymerization temperature. Here we assume that the cure kinetics are identical to the data shown in FIG. 12 at scale and that the cure temperature was the exotherm maximum shown in FIG. 14. Aspen Plus was used to calculate the heat required for polymerization at these different curing temperatures with the assumption of adiabatic conditions, or that we weren't losing a massive amount of heat to the surroundings. Using the experimental results (FIG. 13), we calculated the heat flow profiles for different cure temperatures (FIG. 17), in which positive heat flow indicates an energy input for heating and negative heat flow indicates that the polymerization system is producing excess heat. Important to note is that we assume there is no need to implement cooling.

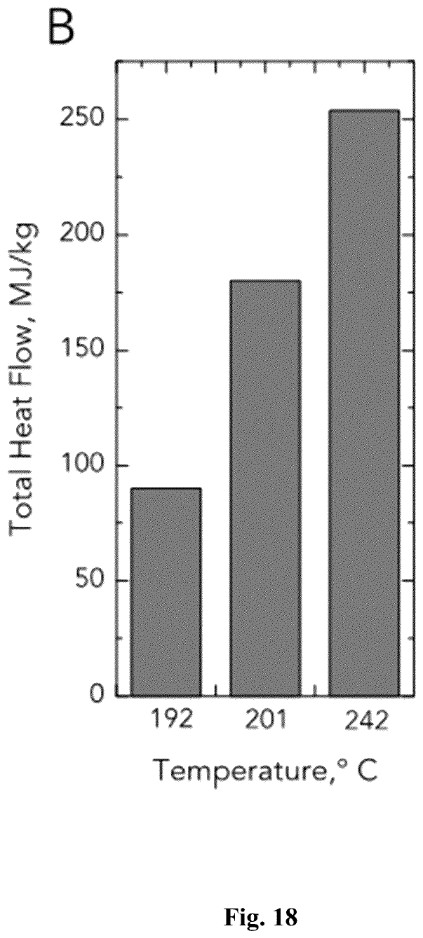

Here we observe that a reduced curing temperature significantly reduces the heat input required. The petrochemical based naphthoxazine, 2b, cures at 242° C., but as the partially bio-based 2a is added in at 5 and 10 mol % the cure temperature is reduced to 201° C. and 192° C. respectively. By modelling the heat flow as a function of time for this reaction (FIG. 17), it is possible to quantify how much energy is required for the reaction by integrating the positive heat flow (FIG. 18). Not surprisingly, curing at higher temperatures requires a higher energy input and integration of the heat flows with respect to time indicates that curing at 201° C. the energy input is 29% lower than the same polymerization at 240° C. Similarly, curing at 192° C. is 50% less energy-intensive than curing at 240° C.

### DISCUSSION

Bio-based molecules off the promise of providing performance advantages relative to petroleum-derived materials due to their unique functionality. These performance advantages in turn could begin to enable the bioeconomy and sustainable materials. Improved performance of materials is directly facilitated by the inherent functionality of bio-based molecules often in the form of heteroatom incorporation such as C—O and C—N.

The bio-based naphthoxazine synthesized in this work utilized a bio-based bifunctional aromatic amine to ring-close the naphthoxazine containing a carboxylic acid end group which reduced the ring opening polymerization temperature by acid-catalyzed ring opening. The inclusion of an acidic proton to promote acid-catalyzed ring opening reduced the cure temperature of two aromatic oxazine systems by 40° C. or greater when used as a 5 mol % additive. Though the ring opening of these materials was dramatically reduced, the overall material exhibits decarboxylation over the course of curing which could lead to voids in the overall material.

However, bio-based naphthoxazine shows promise as an effective acid-catalyzed ring-opening additive for other benzoxazine materials. The petroleum BPA-a benzoxazine was used as a baseline for evaluating the inclusion of the catalytic naphthoxazine and maintaining thermal properties, which was demonstrated through the retention of a T_{g} of 154° C. while reducing the polymerization temperature by 40° C. The material described herein is a mononaphthoxazine system, which does not lead to high crosslinking of the pure polynaphthoxazine.

The use of oxygenated monomers from biomass for polybenzoxazine applications have become more prolific and demonstrate superior performance advantages to petroleum incumbent materials beyond being bio-based. These examples include latent catalytic behavior through hydrogen bonding with exposed phenols, increased crosslinking through allyl alkyl functionality leading to elevated thermomechanical properties, and molecular tunability through both rigid and aliphatic components to improve processability.

The direct performance advantage achieved by reducing the processing energy through the reduced curing temperature was directly facilitated by the unique monomer structure, demonstrated to be bio-derivable. This promising amine structure incorporates several chemical characteristics attractive for oxazine synthesis such as aromaticity for additional rigidity and thermal stability, the renewably sourced primary amine for the oxazine ring formation, and catalytic functionality through a primary alcohol or carboxylic acid which has demonstrated lower polymerization temperatures for benzoxazine systems. Through biosynthesis, the amine monomer requires no additional synthetic modifications and the single-step synthesis of a naphthoxazine with catalytic functionality in the form of a carboxylic acid produced significantly reduced ring-opening polymerization temperatures. The incorporation of bio-based amine production and use can contribute to the reduction of both supply chain energy and green house gas (GHG) emissions.

Reducing the ring-opening polymerization temperature of benzoxazine materials can reduce energetic intensity of the curing process as well as widen the application of these materials as molecular flexibility of starting monomeric components continues to evolve. The implication of bio-based starting materials for these applications provides performance advantages for the polynaphthoxazine such as the 2-(4-aminophenyl) acetic acid replacing a typical petroleum-derived amine such as aniline, can reduced the processing of this material by 100° C. The true performance advantage seen with 2a is as an additive to reduce the polymerization conditions for both 2b as well as BPA-a benzoxazine, which has been used for composite panels in airplanes.⁶⁶ Through modeling the curing process, the 40° C. reduction in cure temperature of BPA-a benzoxazine with 5 mol % naphthoxazine produced a 30% reduction in the energy cost of the processing of the material. The energy savings from lower curing temperatures leads to shorter cycle times for processing materials, higher production of the overall product, and improved revenue.

### CONCLUSIONS

A stable carboxylic acid containing naphthoxazine was designed from a bio-based amine and demonstrated reduced activation energies suited for reduced polymerization temperatures and less intensive curing conditions. The carboxylic acid did not display catalytic behavior at ambient or elevated temperatures below the melting point which is a testament to the shelf-life capabilities of this small molecule. Further, the demonstration of the naphthoxazine as an effective additive to reduce polymerization temperatures was exhibited and can be used as a platform for developing low curing aromatic oxazines from bio-based resources. The biological production of 2-(4-aminophenyl) acetic acid via fermentation from sugars demonstrates the overall net benefits of bio-based amine production through reduced supply chain energies and GHG emissions, as well as direct net energetic reductions in the processing of materials through analysis of both monomer and thermoset production.

### Materials and Methods

Materials. 2-naphthol (99%), paraformaldehyde (95%), 2-(4-aminophenyl) acetic acid (98%), 2-(4-aminophenyl) ethyl alcohol (98%), aniline (>99.5%), and anhydrous 1,4-dioxane (99.8%) was purchased from Sigma Aldrich and used as received. Deuterated DMSO-d₆ was purchased from Cambridge Isotopes.

Synthesis of 2-(4-(1H-naphtho[1,2-e][1,3]oxazin-2 (3H)-yl) phenyl) acetic acid (2a). 2-naphthol (1 mmol), 4-aminophenylacetic acid (1a, 1 mmol), and paraformaldehyde (2 mmol) were refluxed in anhydrous 1,4-dioxane (50 mL) at 110° C. for 6 h. The solution was added dropwise to DI water where the naphthoxazine was precipitated out of the solvent. The obtained yellow powder was recrystallized a second time from dioxane to yield 75% yellow solid (FIG. 4). ¹H NMR (400 MHz, DMSO-d₆, ppm): δ=3.43 (s, 2H, —CH₂—COOH), 4.96 (s, 2H, Ar—CH₂—N), 5.50 (s, 2H, N—CH₂—O), 7.02 (d, 1H), 7.10-7.15 (m, 4H), 7.38-7.42 (m, 1H), 7.53-7.57 (m, 1H), 7.71 (d, 1H), 7.84 (d, 1H), 7.88 (d, 1H), 12.17 (bs, 1H—COOH) (FIG. 5). ¹³C NMR (400 MHz, DMSO-d₆, ppm): δ=39.4, 47.8, 79.8, 113.9, 118.4, 119.3, 122.5, 124.6, 127.7, 128.1, 128.9, 129.4, 130.9, 132.0, 147.7, 152.7, 173.8 (FIG. 6). FT-IR (KBr): ν=1693 cm⁻¹ (C—O stretch), 1236 cm⁻¹ (C—O—C asymmetric stretching), 1143 cm⁻¹ (C—N—C asymmetric stretching), 934 cm⁻¹ (oxazine-ring mode).

Synthesis of 2-(4-(1H-naphtho[1,2-e][1,3]oxazin-2(3H)-yl)phenyl)ethan-1-ol (2c). Similar procedure of 2a followed for production of 2c. 2-naphthol (1 mmol), 4-aminophenylethyl alcohol (1c, 1 mmol), and paraformaldehyde (2 mmol) were refluxed in anhydrous 1,4-dioxane (50 mL) at 110° C. for 6 h. The solution was precipitated into DI water to produce a yellow solid. The yellow solid was crystallized again from dioxane to yield 45%, 1H NMR (400 MHZ, DMSO-d₆, ppm): δ=2.60 (t, 2H, Ar—CH₂—CH₂), 3.05 (q, 2H, —CH₂—CH₂—OH), 4.51 (t, —OH), 4.95 (s, 2H Ar—CH₂—N), 5.49 (s, 2H, N—CH₂—O), 7.01 (d, 1H), 7.06-7.11 (m, 4H), 7.37-7.41 (m, 1H), 7.52-7.56 (m, 1H), 7.71 (d, 1H), 7.83 (d, 1H), 7.87 (d, 1H). ¹³C NMR (400 MHZ, DMSO-d₆, ppm): δ=39.2, 48.0, 63.3, 79.9, 113.9, 118.5, 119.4, 122.4, 124.5, 127.7, 128.9, 129.4, 130.5, 131.9, 132.7, 147.2, 152.7. FT-IR (KBr): ν=3294 (—OH stretching), 2954-2865 (C—H alkane stretch), 1226 cm⁻¹ (C—O—C asymmetric stretching), 1184 cm⁻¹ (C—N—C asymmetric stretching), 935 cm⁻¹ (oxazine-ring mode).

Characterization. The chemical structure of naphthoxazines 2a-c were confirmed by ¹H, ¹³C, and two-dimensional (2D) a Bruker Advance III HD 400 MHZ NMR spectrometer using DMSO-d₆. The relaxation time used for proton NMR integration of resonances was 10 s and an average number of transient scans was 16. The ¹³C NMR spectrum was also acquired in DMSO-de at a frequency of 100 MHz with an average number of transient scans of 256. The 2D ¹H-¹³C HSQC NMR was conducted with 1024 points, sweep width 16 ppm in the F2 dimension and 256 data points, 300 ppm sweep width for the F1 dimension with a 1.5 s relaxation delay and 256 total scans. Further structural characterization was completed with a Nicolet iS50 FTIR spectrophotometer with a single reflectance ATR detector. Approximately 1-5 mg of naphthoxazine was ground with KBr, and the spectra were collected in the range of 4000-650 cm 1 at room temperature. Thermal decomposition was studied using a TA Instruments Q-500 thermal gravimetric analyzer at a heating rate of 5° C./min with a nitrogen flow of 60 mL/min up to 550° C. A TA instruments Q-5000 digital scanning calorimeter (DSC) was utilized at with a heating rate of 5° C./min and a nitrogen flow rate of 60 mL/min. The activation energy was calculated by analyzing samples (2.5±0.5 mg) at different heating rates of 2, 5, 10, 15, and 20° C./min in sealed hermetic aluminum pans.

The invention may be further understood by the following non-limiting examples:

Example 1. A method comprising:
providing a bio-derived aromatic amine;
reacting the bio-derived aromatic amine with a phenol to generate a naphthoxazine.

Example 2. The method of example 1 further comprising polymerizing the naphthoxazine to form a polybenzoxazine.

Example 3. The method of example 2, wherein the step of polymerizing is performed via acid-catalyzed cationic ring opening polymerization.

Example 4. The method of example 2, wherein the step of polymerizing is performed via amine protection/deprotection.

Example 5. The method any of examples 2-4, wherein the step of polymerizing is performed at a temperature less than or equal to 300° C.

Example 6. The method of any of examples 2-5, wherein the step of polymerizing is performed in the presence of formaldehyde.

Example 7. The method of any of examples 1-6, wherein the phenol is a bio-based phenol.

Example 8. The method of any of examples 1-7, wherein the phenol is selected from the group of naphthol, resveratrol, resorcinol, cardanol, guaiacol, eugenol, thymol, vanillin, sesamol, terpenediphenol, magnolol, daidzein, naringenin, urushiol, functionalized coumarin, naringenin, apigenin or a combination thereof.

Example 9. The method of any of examples 1-8, wherein the phenol comprises naphthol.

Example 10. The method of any of examples 1-9, wherein the aromatic amine is generated microbially from sugar.

Example 11. The method of any of examples 1-10, wherein the aromatic amine is defined by the formula [FX1]; [FX2]; [FX3]; or a combination thereof; wherein R is a nitrogen protecting group comprising an alcohol, a carboxylic acid, BOC (tert-butyloxycarbonyl), a phthalic anhydride, a tehtrachlorophthalic anhydride, or a combination thereof.

Example 12. The method of example 11, wherein R is a carboxylic acid.

Example 13. The method of any of examples 1-12, wherein the step of reacting is performed at a temperature less than or equal to 200° C.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure. For example, when a device is set forth disclosing a range of materials, device components, and/or device configurations, the description is intended to include specific reference of each combination and/or variation corresponding to the disclosed range.

Every formulation or combination of components described or exemplified herein can be used to practice the invention, unless otherwise stated.

Whenever a range is given in the specification, for example, a density range, a number range, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter is claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method comprising:
providing a bio-derived aromatic amine;
reacting the bio-derived aromatic amine with a phenol to generate a naphthoxazine.

2. The method of claim 1 further comprising polymerizing the naphthoxazine to form a polybenzoxazine.

3. The method of claim 2, wherein the step of polymerizing is performed via acid-catalyzed cationic ring opening polymerization.

4. The method of claim 2, wherein the step of polymerizing is performed via amine protection/deprotection.

5. The method claim 2, wherein the step of polymerizing is performed at a temperature less than or equal to 300° C.

6. The method of claim 2, wherein the step of polymerizing is performed in the presence of formaldehyde.

7. The method of claim 1, wherein the phenol is a bio-based phenol.

8. The method of claim 1, wherein the phenol is selected from the group of naphthol, resveratrol, resorcinol, cardanol, guaiacol, eugenol, thymol, vanillin, sesamol, terpenediphenol, magnolol, daidzein, naringenin, urushiol, functionalized coumarin, naringenin, apigenin or a combination thereof.

9. The method of claim 1, wherein the phenol comprises naphthol.

10. The method of claim 1, wherein the aromatic amine is generated microbially from sugar.

11. The method of claim 1, wherein the aromatic amine is defined by the formula: 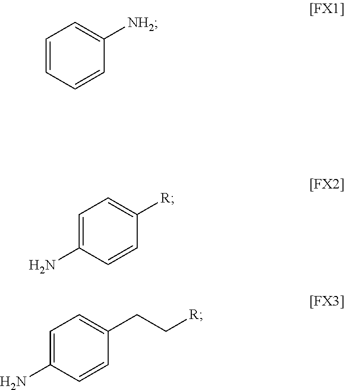
or a combination thereof;
wherein R is a nitrogen protecting group comprising an alcohol, a carboxylic acid, BOC (tert-butyloxycarbonyl), a phthalic anhydride, a tehtrachlorophthalic anhydride, or a combination thereof.

12. The method of claim 11, wherein R is a carboxylic acid.

13. The method of claim 1, wherein the step of reacting is performed at a temperature less than or equal to 200° C.
